(19) United States
(12) Patent Application Publication
(10) Pub. No.: US 2004/0152102 A1
(43) Pub. Date: Aug. 5, 2004

(54) Use of transcription factor nak-1 or genes regulated by transcription factor nak-1 for the diagnosis and/or therapy of inflammatory and malignant diseases

(76) Inventors: Bernd R Binder, Vienna (AT); Johannes Schmid, Modling (AT); Johannes Breuss, Vienna (AT); Peter Hufnagl, Vienna (AT); Florian Gruber, Vienna (AT)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/482,207

(22) PCT Filed: Jun. 27, 2002
(86) PCT No.: PCT/AT02/00188

(51) Int. Cl. C12Q 1/70; C12Q 1/68; G01N 33/53

(52) U.S. Cl. 435/6; 435/5; 435/7.1

(57) ABSTRACT

Interaction of the PAI-1 promotor region with NAK-1 was twice detected using a genetic screen.

NAK-1 is up-regulated in an NFκI dependent mechanism in many kinds of inflammation reaction and in others, however, it is not, but stimulates in both cases the expression of PAI-1.

In the inflammation reaction the up-regulation of PAI-1 is followed by that of NAK-1 and increased NAK-1 binding to the PAI-1 promotor is detectable.

NAK-1 is up-regulated in atherosclerotic vessels where PAI-1 is also strongly expressed in the same cells.

### STATE OF THE ART

In inflammatory conditions in human or animal organisms, transcription factors play a decisive role. These proteins, which can bind to DNA and thus influence the regulation of their target genes carry information as to the internal state of the cell as well as information as to the environment of the cell or factors which can bind to the cell and further to the gene and which thus can react to these states or state changes.

One transcription factor which assumes central functions in inflammatory states, is NFkB—a protein which upon activation of the cell through inflammation medicators like IL-1, TNF or LPS is transported into the cell nucleus and can switch on “target genes”. These genes contain in their control region binding sites for NFkB; signals are given upon contact of the protein with these binding sites. The production of these target genes should be produced in high number as the answer of the cell to the inflammation stimulus.

However, not all responses of the cell to inflammation stimuli are given by the transcription factor NFkB, since several genes also achieve high levels even if they are not activated through a NFkB binding site. In these cases, the up regulation of the genes can be explained in that either other transcription factors react directly to the inflammation stimuli and then bind to these genes or that further transcription factors are indicated as a secondary response of NFkB and bring about an up regulation through secondary target genes on their behalf. These processes can also arise simultaneously in the regulation of certain genes. The group of genes which are turned on indirectly through secondary NFkB induced transcription factors can possibly be an important subgroup of genes that are first turned on in a delayed manner and possibly are effective to modulate the inflammation conditions.

An important gene for inflammation processes and also as a trigger for atherosclerosis is the plasminogen activator inhibitor 1, PAI-1.

The PAI-1 protein is a key factor for the control of fibrin deposits in and around blood vessels. In addition it regulates the formation and the decomposition of the extra cellular matrix and is thus involved in plastic modifications of the tissue in the region of the blood vessels. PAI-1 also plays a role in tumor processes since PAI-1 is correlated with the malignant character of tumors and is associated with the formation of metastases.

Several research groups, based upon patient material and investigations of cell cultures, have found that PAI-1 is up-regulated in atherosclerotic vessels and can be stimulated by inflammation mediators like TNFα, LPS and IL-1. There are no NFkB binder sites in regulation regions of PAI-1 and thus one of the above mentioned alternative mechanisms must be activated to up-regulate the PAI-1 in inflammation processes.

### Discovery of the Transcription Factor NAK-1 as Inflammation Mediators

To clarify the mechanism of the stimulation of PAI-1 by inflammation mediators, a “genetic screen” was carried out to identify the proteins which interact with certain section of the PAI-1 regulation regions. As “coder” the region −250 to −270 from the transcription start was used since reporter gene analysis indicated there there were regulation elements for PAI-1 in the inflammation reaction.

Unexpectedly we were able to identify the transcription factor NAK1 as interaction partners in two independent clones in this DNA region. NAK1 (NR4A1) is the first member of the “Nuclear Receptor Subfamily 4/Group A”; the homologous gene in the mouse and rat were identified as Nur77 or NGFI-B. First identified was N10 of Ryseck, et al 1989 which was localized on the human chromosome 12 (12q13). Chang et al cloned it in the same year as a further member of the “Steroid Receptor Superfamily” under the name TR3. Nakai et al demonstrated in 1990 that NAK1 is inducible by serum and certain mitogenes and thus lies in the series of genes of the family of “Immediate Early Response Genes”.

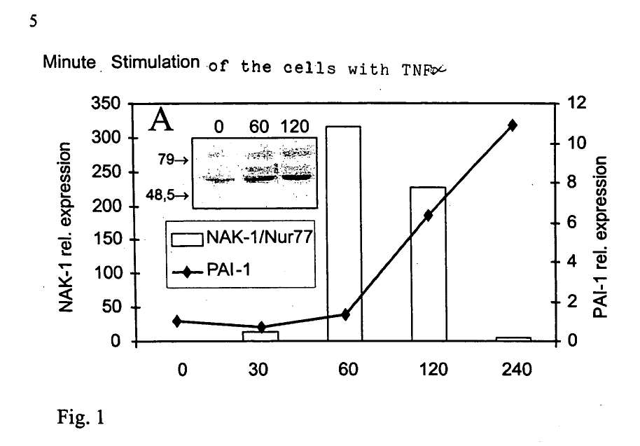

To detect whether NAK-1 also can be induced by inflammation mediators, endothelial cells with TNFα are activated and NAK-1 mRNA is detected by means of “Relative Quantitative PCR”. FIG. 1 shows that NAK-1 mRNA expression is inducible by TNFα in endothelial cells and that any induction of PAI-1 mRNA follows. In a small window, the induction of the NAK1 protein expression to this stimulus is visible.

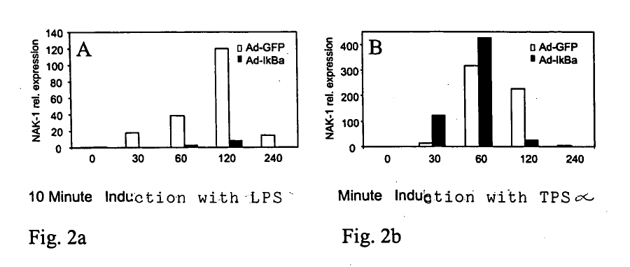

To detect whether NAK-1 in its turn is dependent upon NFκB, endothelial cells were stimulated by inflammation mediators and simultaneously the NFκB activation was blocked by transection with an adeno virus that codes for an inhibitor of NFκB (IkBa). FIG. 2a shows that NAK-1 mRNA is only up-regulated by bacterial toxins (LPS) when, upon inflammation stimulation of the cells, the NKκB signal transduction cascades intact. The inflammation stimulus (LPS) stimulates the expression of NAK1 in untransfixed endothelial cells and endothelial cells infected with control virus but not in such which are transfixed with IkBa and thus do not have NFκB signal transduction. FIG. 2b shows that this mechanism is not active when NAK1 mRNA expression is induced by TNFα. The indication is not blocked by inhibitors of NFkB which suggests two inflammation mechanisms which converge in the expression of NAK-1. This is to be expected that through an inhibition of the NAK-1 function as a transcription activator another spectrum of inflammation reaction genes is blocked when upon an inhibition of NFkB signal transduction path.

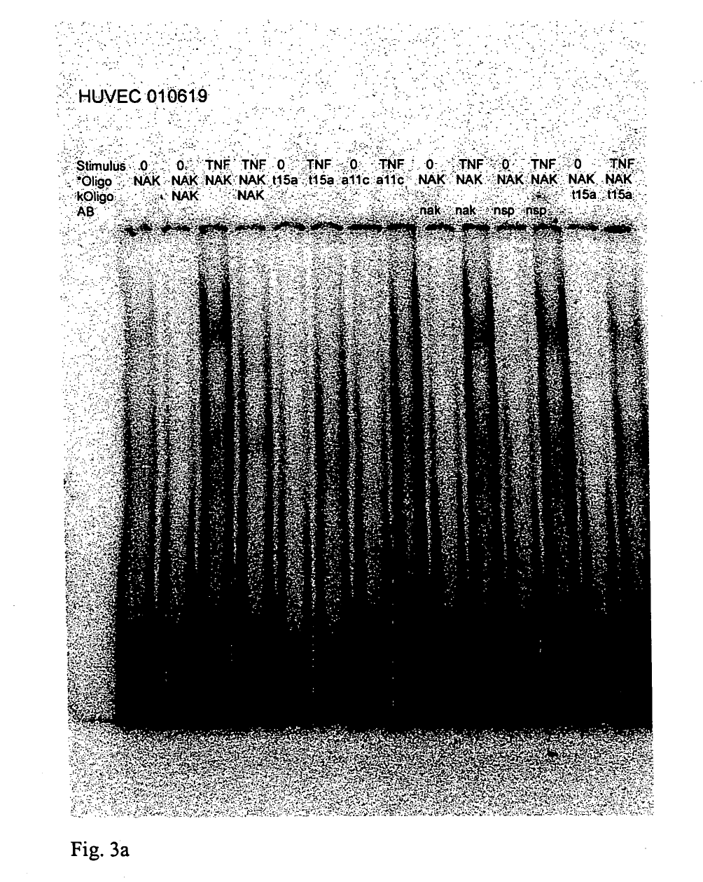

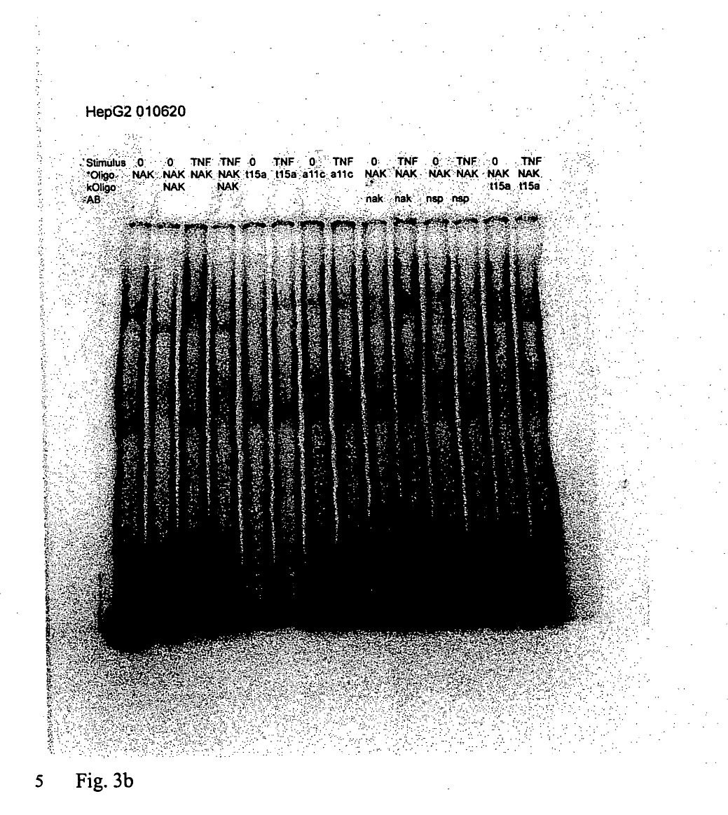

To achieve the detection that in fact NAK-1 binds to a greater extent to the PAI-1 promotor during the infraction reaction, so called electrophoretic mobility shift experiments (EMSA) were carried out. In FIG. 3a it can be seen that a ds oligonucleotide which extends over the region used in the screen, produces a specific band in the EMSA. This band can be blocked by mutation at the consensus binding site. This band can be blocked by mutation of an adenine based 5′ of the consensus binding site which appears to be important for the binding of NAK-1 as a monomer. In this illustration one can see that a nuclear protein binds specifically to the DNA region. Consideration of that mutation of the consensus binding site for NAK1 is blocked by a point mutation of this binder.

It can additionally be seen that this binding activity can be retarded in the gel by the addition of an antibody which binds to NAK-1, thereby forming a further indication for resistance of the necessity of NAK-1 in this binding reaction. An identical result can also be observed in the model cell line HepG2 (FIG. 3b).

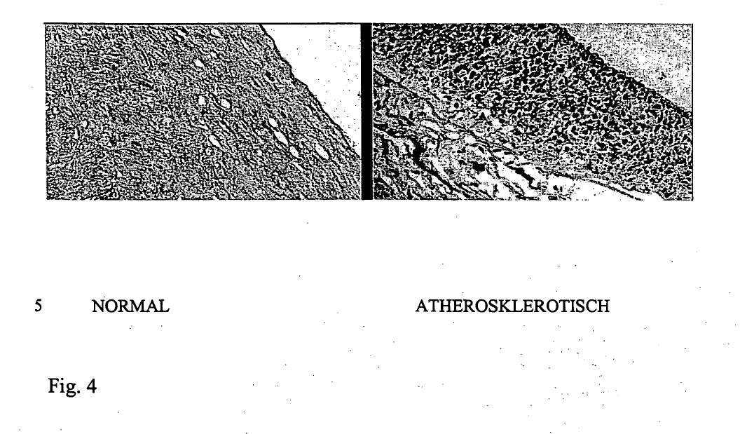

In order to obtain an indication that NAK-1 is up-regulated also in the case of inflammatory conditions (as in the case of atherosclerosis) in humans in vivo, normal and atherosclerotic vessels are colored with an antibody against NAK-1. It has been found that in the atherosclerotic vessels NAK-1 is increasingly expressed while the signal appears to fail in normal vessels (FIG. 4).

FIG. 4 shows on the left side a normal tissue; less NAK-1 antigen is detected. On the right side of FIG. 4 an atherosclerotic tissue is shown whereby the cells which express NAK-1 antigens are colored darkly.

### Conclusions

From this data it can be concluded that NAK-1 is a transcription factor which is only partly NFκB dependent and which can trigger through different inflammation stimuli, secondary genes like for example PAI-1. The known inhibitors of NFκB cannot inhibit these disorders alone and the ability to intervene in the function of NAK-1 as a transcriptional activator constitutes a new and broader possibility for the treatment of inflammatory disorders and their consequence upon the vascular system.

Through the determination of NAK-1 mRNA and protein expression or specific NAK-1 dependent genes, one can obtain information as to expected inflammatory reaction. Similarly one can expect to be able to modulate such inflammation reaction by influencing the NAK-1 induced transcription.

The disclosure and content of the following literature references are components of the present patent application.

### Literature

Chang, C., Kokontis, J., Liao, S. S., and Chang, Y. (1989). Isolation and characterization of human TR3 receptor: a member of steroid receptor superfamily. J. Steroid Biochem. 34, 391-395.

Chomiki, N., Henry, M., Alessi, M. C., Anfosso, F., and Juhan-Vague, I. (1994). Plasminogen activator inhibitor-1 expression in human liver and healthy or atherosclerotic vessel walls. Thromb. Haemost. 72, 44-53.

Christ, G., Hufnagl, P., Kaun, C., Mundigler, G., Laufer, G., Huber, K., Wojta, J., and Binder, B. R. (1997). Antifibrinolytic properties of the vascular wall. Dependence on the history of smooth muscle cell doublings in vitro and in vivo. Arterioscler. Thromb. Vasc. Biol. 17, 723-730.

de Martin, R., Hoeth, M., Hofer-Warbinek, R., and Schmid, J. A. (2000). The transcription factor NF-kappa B and the regulation of vascular cell function. Arterioscler. Thromb. Vasc. Biol. 20, E83-E88.

Emeis, J. J. and Van den Hoogen, C. M. (1992). Pharmacological modulation of the endotoxin-induced increase in plasminogen activator inhibitor activity in rats. Blood Coagul. Fibrinolysis 3, 575-581.

Healy, A. M. and Gelehrter, T. D. (1994). Induction of plasminogen activator inhibitor-1 in HepG2 human hepatoma cells by mediators of the acute phase response. J. Biol. Chem. 269, 19095-19100.

Nakai, A., Kartha, S., Sakurai, A., Toback, F. G., and DeGroot, L. J. (1990). A human early response gene homologous to murine nur77 and rat NGFI-B, and related to the nuclear receptor superfamily. Mol. Endocrinol. 4, 1438-1443.

Oitzinger, W., Hofer-Warbinek, R., Schmid, J. A., Koshelnick, Y., Binder, B. R., and de Martin, R. (2001). Adenovirus-mediated expression of a mutant IkappaB kinase 2 inhibits the response of endothelial cells to inflammatory stimuli. Blood 97, 1611-1617.

Ryseck, R. P., Macdonald-Bravo, H., Mattei, M. G., Ruppert, S., and Bravo, R. (1989). Structure, mapping and expression of a growth factor inducible gene encoding a putative nuclear hormonal binding receptor. EMBO J. 8, 3327-3335.

Schneiderman, J., Sawdey, M. S., Keeton, M. R., Bordin, G. M., Bernstein, E. F., Dilley, R. B., and Loskutoff, D. J. (1992). Increased type 1 plasminogen activator inhibitor gene expression in atherosclerotic human arteries. Proc. Natl. Acad. Sci. U.S.A 89, 6998-7002.

van Hinsbergh, V. W., Kooistra, T., van den Berg, E. A., Princen, H. M., Fiers, W., and Emeis, J. J. (1988). Tumor necrosis factor increases the production of plasminogen activator inhibitor in human endothelial cells in vitro and in rats in vivo. Blood 72, 1467-1473.

1. The use of NAK-1 mRNA, proteins or antibodies for the detection of NAK-1, or of mRNA and proteins of the gene which regulates transcription through NAK-1 for the detection of an inflammation reaction and its detrimental effects on the vasculature in the molecular or transcriptional and protein expression plane.

2. The use of NAK-1 mRNA proteins or antibodies for the detection of NAK-1 or of mRNA and proteins of the gene which regulates transcription through NAK-1 for the detection of atherosclerosis in the molecular or transcriptional or protein expression plane.

3. The use of NAK-1 and dominant negative mutants of this protein, (as plasmid-DNA, as fusion proteins with a transducing peptide or packed in adeno viral or retroviral gene transfer vehicle) in the treatment of an inflammation reaction.

4. The use of NAK-1 and dominant negative mutants of this protein, (as plasmid-DNA, as fusion proteins with a transducing peptide or packed in adeno viral or retroviral gene transfer vehicle) in the treatment of an atherosclerosis.
